(19) United States
(12) Patent Application Publication
(10) Pub. No.: US 2019/0328716 A1
(43) Pub. Date: Oct. 31, 2019

(54) LIPOSOMAL FORMULATIONS OF AMIDINE SUBSTITUTED BETA-LACTAM COMPOUNDS FOR USE IN THE TREATMENT OF BACTERIAL INFECTIONS

(71) Applicant: AICURIS ANTI-INFECTIVE CURES GMBH, Wuppertal (DE)

(72) Inventor: Yogeshwar BACHHAV, Mumbai (IN)

(73) Assignee: AICURIS ANTI-INFECTIVE CURES GMBH, Wuppertal (DE)

(21) Appl. No.: 16/462,009

(22) PCT Filed: Nov. 17, 2017
(86) PCT No.: PCT/EP2017/079644

§ 371 Date: May 17, 2019

(30) Foreign Application Priority Data
Nov. 18, 2016 (EP) 16199658.2

(51) Int. Cl. A61K 31/427; A61K 9/127

(52) U.S. Cl. CPC A61K 31/427; A61K 9/127

(57) ABSTRACT

The present invention provides new and effective liposomal formulations for the administration of beta-lactams.

### FIELD OF THE INVENTION

This invention relates to liposomal β-lactam formulations, encapsulation of said β-lactams into liposomes for drug delivery purposes and the use of said formulations in the treatment and prophylaxis of bacterial infections.

### BACKGROUND

This invention relates to novel β-lactam formulations, their preparation and use. In particular, this invention relates to novel β-lactam compounds which are amidine substituted monobactam derivatives useful as antimicrobial agents and their encapsulation into liposomes as drug delivery system.

Public health experts and officials consider the emergence and spread of antibiotic resistant bacteria as one of the major public health problems of the 21st century. Although not a new phenomenon per se, the spread of antibiotic resistant bacteria has reached an unprecedented dimension. While the most resistant isolates continue to emerge in the hospital setting, physicians and epidemiologists are encountering increasing numbers of resistant bacteria in the community among people without previous healthcare contact.

The number of patients who are dying from untreatable nosocomial infections continues to grow. Therapeutic options are especially limited for infections due to multi-drug-resistant Gram-negative pathogens including Enterobacteriaceae and non-fermenters, a situation made worse by the fact that the pipelines of the pharmaceutical industry contain few compounds with promising resistance breaking profiles There is a need to increase the number of effective antimicrobial drugs to defeat infections caused by bacteria that have become resistant to existing medicines (Jim O'Neill; The Review on antimicrobial resistance; Tackling drug-resitant infections globally, 2016).

The highly successful and well-tolerated class of β-lactam antibiotics has historically been one mainstay for the treatment of infections caused by Gram-negative pathogens. Among these especially 3rd-generation cephalosporins, carbapenems and monobactams are extensively used for the treatment of infections with Gram-negative bacteria. However, a vast array of more than 1000 β-lactamases and further resistance mechanisms severely endanger the midterm usability of the current compounds in these subclasses. Especially extended-spectrum β-lactamases (ESBLs) and carbapenemases are important drivers of resistance. New β-lactams with resistance breaking properties are urgently needed to fill the gap.

Novel β-lactam compounds, which are amidine substituted monobactam derivatives with very promising antibacterial and antimicrobial properties, have been disclosed in WO 2013/110643 recently.

Problems associated with administering drugs are, amongst others, a too early inactivation of the pharmaceutically active ingredient, e.g., by unspecific interactions in the body. This may result in insufficient concentration levels of the therapeutically active compounds. On the other hand, administration of too high concentrations of active ingredients may be associated with adverse effects. Generally, the therapeutic window may be narrow and may be close to either non-effective or potentially toxic doses.

Therefore, it is an objective to provide well-tolerated formulations of β-lactam compounds that can be administered without side effects that are encountered frequently, e.g. when higher amounts of surfactants and/or cosolvents etc. are used, which are not suitable for children on account of their side effects.

Further, another object of the present invention is to provide a liposomal formulation of hydrophilic active compounds as it is particularly difficult encapsulate respective active ingredients in liposomes.

The above objectives have been solved by the encapsulation of the inventive β-lactam compounds into liposomal drug delivery systems.

The advantages of inventive drug delivery systems are their targeted modes of action with reduced toxicity and side effects accompanied by higher efficiency due to the possibility of delivering the pharmaceutically active ingredient to the targeted infected cells or organs, preventing an early metabolization and inactivation of the medication as well as damages of healthy tissue.

In drug delivery, a very successful class of vesicles are liposomes, where the amphiphilic molecule is a phospholipid. In these amphiphiles, the hydrophilic head consists of a negatively charged phosphate or phosphate ester moiety which is connected to two hydrophobic fatty acid chains by their esterification, e.g. with glycerol. In water, phospholipids self-assemble in a way that the interaction of the non-polar hydrophobic fatty acid alkyl tails with water is minimized. Therefore, phospholipids self-organize into bi-layered membranes and liposomes with the hydrophobic alkyl chains inside the corresponding structure and the polar phosphate groups directed towards the aqueous medium. The nature of the phospholipid, i.e. its charge, size and pH dependent properties, mainly influence the characteristics of the resulting liposome, including its therapeutic efficiency, stability and pharmacokinetic properties. [Giuseppina Bozzuto, Agnese Molinari; Liposomes as nanomedical devices; International Journal of Nanomedicine 2015, 10, 975-999]

With their morphology and structure very similar to cell membranes, liposomes are highly biocompatible, easily biodegradable, non-toxic materials and hence perfect candidates for drug-delivery. Liposomes consist of three distinct compartments for drug encapsulation. Depending on its solubility properties, the drug is either located in the aqueous core, is intercalated in the lipid bilayer or is attached to the inner and outer polar interfaces of the phospholipid with water. Thus, hydrophobic as well as hydrophilic drug may be encapsulated into liposomes. Polar and water-soluble drugs are dissolved or dispersed in the inner aqueous compartment or located near the inner and outer polar head groups, whereas hydrophobic drugs are intercalated between the hydrophobic alkyl chains of the phospholipid bilayer.

In order to stabilize the bilayer structure of liposomes and to trigger the bilayer density, which is an important factor if it comes to the release rates of the encapsulated compound, steroids may be added to the phospholipid bilayer. A naturally occurring steroid is cholesterol, which increases the stiffness and hence the mechanical stability of phospholipid bilayers on account of its rigid structure. Cholesterol intercalates between the hydrophobic alkyl chains in the core of the bilayer and hence reduces the permeability of the liposome. Moreover, cholesterol is applied to attach molecules to the surface of liposomes, equipping the drug delivery system with sensing, stimuli-responsive or “stealth” properties.

Methods of liposome preparation and drug encapsulation include the thin-film hydration method, reverse-phase evaporation method, detergent-depletion method, and the dehydration-rehydration method. Among these established methods, the most convenient one for liposomal drug encapsulation is the thin-film hydration method, where a lipid is dissolved in an organic solvent. The thin-film that remains after complete removal of the organic solvent is then hydrated with an aqueous solution/buffers, resulting in the formation of liposomes.

The above-mentioned β-lactam compound shows pH dependent solubility that is poorly soluble at neutral and alkaline pH, while highly soluble at acidic pH. Its also highly soluble in water/acetonitrile mixtures. However, both strong acidic pH and acetonitrile are not desirable for pharmaceutical drug product formulations. Surprisingly it was found that active loading of β-lactam compounds into the liposomes is preferably performed by a trans-membrane gradient.

### DETAILED DESCRIPTION OF THE INVENTION

The present invention thus relates to:

A liposomal pharmaceutical formulation comprising a compound according to formula (I) as active ingredient,

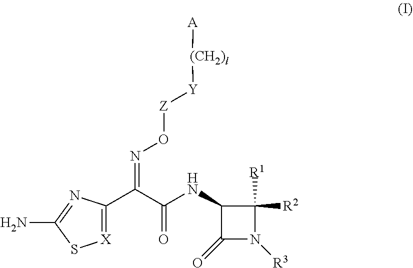

characterized in that

R¹ and R² represent methyl,

R³ represents —O—(SO₂)OH,

X represents CH,

Z represents a two carbon alkyl-chain, substituted with a carboxy substituent,

Y represents O,

l represents 0

A represents phenyl substituted with a substituent of the following formula

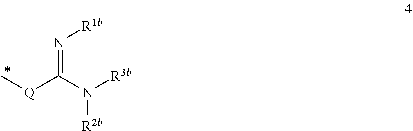

wherein R^{1b} and R^{2b} represent hydrogen, R^{3b} represents aminoethyl, azetidine, pyrrolidine or piperidine, Q represents a bond, * is the linkage site to the residue represented by A, and the salts thereof, the solvates thereof and the solvates of the salts thereof, wherein the liposomes comprise at least one phospholipid and one steroid.

In a liposomal pharmaceutical formulation as defined above, the active ingredient may be at least one member of the group of compounds selected from formulae (Ia) to (Ig):

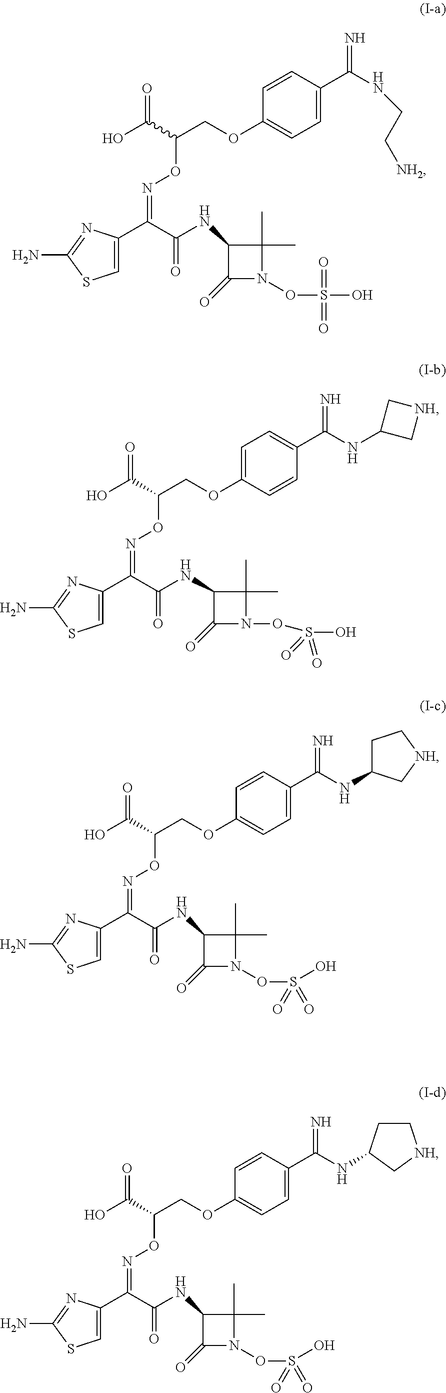 [SMILES: CC1(C)[C@H](NC(=O)/C(=N\OC(COc2ccc(C(=N)NCCN)cc2)C(=O)O)c2csc(N)n2)C(=O)N1OS(=O)(=O)O ; CC1(C)[C@H](NC(=O)/C(=N\O[C@@H](COc2ccc(C(=N)NC3CNC3)cc2)C(=O)O)c2csc(N)n2)C(=O)N1OS(=O)(=O)O ; CC1(C)[C@H](NC(=O)/C(=N\O[C@@H](COc2ccc(C(=N)N[C@H]3CCNC3)cc2)C(=O)O)c2csc(N)n2)C(=O)N1OS(=O)(=O)O ; CC1(C)[C@H](NC(=O)/C(=N\O[C@@H](COc2ccc(C(=N)N[C@@H]3CCNC3)cc2)C(=O)O)c2csc(N)n2)C(=O)N1OS(=O)(=O)O]

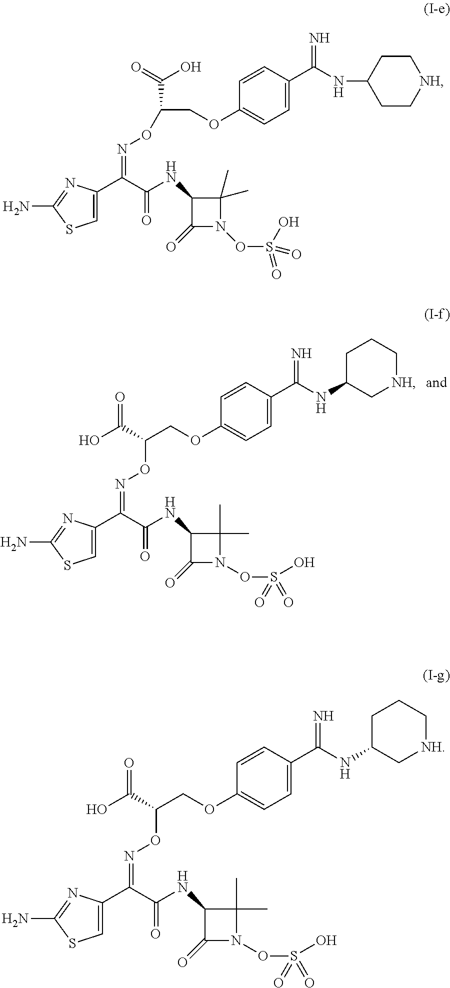 [SMILES: CC1(C)[C@H](NC(=O)/C(=N\O[C@@H](COc2ccc(C(=N)NC3CCNCC3)cc2)C(=O)O)c2csc(N)n2)C(=O)N1OS(=O)(=O)O ; CC1(C)[C@H](NC(=O)/C(=N\O[C@@H](COc2ccc(C(=N)N[C@H]3CCCNC3)cc2)C(=O)O)c2csc(N)n2)C(=O)N1OS(=O)(=O)O ; CC1(C)[C@H](NC(=O)/C(=N\O[C@@H](COc2ccc(C(=N)N[C@@H]3CCCNC3)cc2)C(=O)O)c2csc(N)n2)C(=O)N1OS(=O)(=O)O]

In embodiments of the invention, the liposomal pharmaceutical formulation comprises a compound of formula (I-g).

In further embodiments of the present invention, the liposomal pharmaceutical formulation comprises a phospholipid selected from the group comprising phosphotidylcholine, phosphotidylserine, Phosphotidylethanolamine, phosphoinositol, 1,2-dilauroyl-sn-Glycero-3-Phosphocholine, 1,2-dioleoyl-sn-Glycero-3[Phospho-L-Serine] sodium salt, dipalmitoylphosphotidylcholine, distearoylphosphotidylcholine, dipalmitoylphosphotidylserine, dipalmitoylphosphotidylglycerol, 1-stearoyl-2-linoleoyl-sn-glycero-3-[phosphor-L-serine] sodium salt, dioleoylphosphotidylcholine, and sphingomyelin.

In further embodiments of the present invention, pharmaceutical formulation as defined above, wherein the steroid is selected from the group comprising cholesterol, derivatives of cholesterol, and polymer-derivatized cholesterol.

The liposomal formulation according to the present invention may also contain other steroid components such as derivatives of cholesterol, coprostanol, cholestanol, or cholestane, and combinations of PC and cholesterol. They may also or alternatively contain organic acid derivatives of sterols such as cholesterol hemisuccinate (CHS) e.g. cholesterol hemisuccinate, Suitable sterols for incorporation in the liposomes include cholesterol, cholesterol derivatives, cholesteryl esters, vitamin D, phytosterols, steroid hormones, and mixtures thereof. Useful cholesterol derivatives include cholesterol phosphocholine, cholesterol-polyethylene glycol, and cholesterol-SO4, while the phytosterols may be sitosterol, campesterol, and stigmasterol.

In further embodiments of the present invention, the liposomal pharmaceutical formulation comprises a phospholipid, wherein the phospholipid is distearoylphosphotidylcholine.

In further embodiments of the present invention, the liposomal pharmaceutical formulation comprises a steroid is cholesterol.

In further embodiments of the present invention as defined above, the ratio of phospholipid to steroid is in the range from 60:40 to 40:60.

In further embodiments of the present invention as defined above, the liposomal pharmaceutical formulation comprises a ratio of phospholipid to steroid that is 55:45.

In further embodiments of the present invention as defined above, the lipid to drug ratio is in the range of 1:0.1 to 1:0.5.

In further embodiments of the present invention as defined above, the lipid to drug ratio is 1:0.3.

In further embodiments of the present invention as defined above, the range of particle sizes is 25-750 nm, 50-500 nm, 75-300 nm, e.g. 100-250 nm, 120-200 nm, or 130-180.

In further embodiments of the present invention as defined above, the liposomal pharmaceutical formulation according to any one of the preceding embodiments is for use in the treatment and/or prevention of bacterial infections.

The present invention provides liposomal formulations comprising at least one of the compounds of formulae (I) and (Ia to I-g), e.g. a compound of formula (I-g). These liposomal formulations are particularly well-suited in the use or in methods of the treatment and/or prevention of intracellular bacterial infections, because conventional (e.g. non-liposomal compounds show either a poor intracellular diffusion and retention or reduced activity at acidic pH of the lysosome. Further, the liposomal formulations comprising at least one of the compounds of formulae (I) and (Ia to I-g), e.g. a compound of formula (I-g) have a higher efficacy because they are well-tolerated at pharmaceutical relevant dosages permitting the administration on a once-a-day schedule without loss in therapeutic efficacy. However, it is also possible to administer the formulations according to the present invention more often, e.g. two or three times a day or more often. More frequent dosing allows for more easily maintaining a constant level of a therapeutically active concentration of the herein disclosed compounds over time (e.g. the treatment period).

The herein described liposomal formulations also permit reducing the overall dose of the active compounds, which is related to the improved stability and the mode of action against intracellular bacteria. Consequently, adverse effects associated with administration of the antibiotic compounds disclosed herein can be reduced. Therefore, these liposomal formulations are advantageously particularly well-suited for treatment of paediatric patients. For example, it will be possible to administer a dose of 0.5 g of the herein described compounds (I-a) to (I-g) with a 500 ml infusion. Further, liposomal formulations can also improve the oral absorption of poorly permeable drugs such as the herein described compounds (I-a) to (I-g), e.g. (I-g). Liposomal formulations for oral administration comprising at least one of the herein described compounds (I-a) to (I-g), e.g. compound (I-g) are therefore provided. These formulations are also particularly well-suited for paediatric uses as this mode of administration does not induce fear and painful experiences in children.

Advantageously, liposomal formulations of the herein disclosed compounds of formulae (I) and (Ia to I-g), e.g. a compound of formula (I-g), permit an improved treatment of antibiotic resistant bacterial strains, because liposomal formulations increase the sensitivity of such resistant bacteria (cf. e.g. Lagacé, J., et al.; J. Microendcapsul. 1991 January-March; 8 (1):53-61).

The present invention relates also to a method of treatment and/or prevention of bacterial infection and/or spread of bacterial infection comprising administering a lipid formulation according to any one of the preceding embodiments.

The compounds of formulae (I) and (Ia to I-g) for use according to the invention may, depending on their structure, exist in stereoisomeric forms (enantiomers, diastereomers). The invention therefore also encompasses the enantiomers or diastereomers and respective mixtures thereof. The stereoisomerically uniform constituents can be isolated in a known manner from such mixtures of enantiomers and/or diastereomers.

If the compounds of formulae (I) and (I-a to I-g) for use according to the invention occur in tautomeric forms, the present invention encompasses all tautomeric forms.

Salts preferred for the purposes of the present invention are physiologically acceptable salts of the compounds of formulae (I) and (I-a to I-g) for use according to the invention. Also encompassed however are salts which are themselves not suitable for pharmaceutical applications but can be for use for example for the isolation or purification of the compounds of formulae (I) and (I-a to I-g) for use according to the invention.

Examples of pharmaceutically acceptable salts of the compounds of formula (I) include salts of inorganic bases like ammonium salts, alkali metal salts, in particular sodium or potassium salts, alkaline earth metal salts, in particular magnesium or calcium salts; salts of organic bases, in particular salts derived from cyclohexylamine, benzylamine, octylamine, ethanolamine, diethanolamine, diethylamine, triethylamine, ethylenediamine, procaine, morpholine, pyrroline, piperidine, N-ethylpiperidine, N-methylmorpholine, piperazine as the organic base; or salts with basic amino acids, in particular lysine, arginine, ornithine and histidine. Examples of pharmaceutically acceptable salts of the compounds of formulae (I) and (I-a to I-g) for use according to the invention also include salts of inorganic acids like hydrochlorides, hydrobromides, sulfates, phosphates or phosphonates; salts of organic acids, in particular acetates, formates, propionates, lactates, citrates, fumarates, maleates, benzoates, tartrates, malates, methanesulfonates, ethanesulfonates, toluenesulfonates or benzenesulfonates; or salts with acidic amino acids, in particular aspartate or glutamate.

Solvates of formulae (I) and (I-a to I-g) for use for the purposes of the invention refer to those forms of the compounds of formulae (I) and (I-a to I-g) for use according to the invention which in the solid or liquid state form a complex by coordination with solvent molecules. Hydrates are a specific form of solvates in which the coordination takes place with water.

In another aspect of the present invention, said formulation comprises liposomes, which may be selected from the group comprising conventional liposomes, pH sensitive liposomes, cationic liposomes, immuno-liposomes and long acting liposomes. In one aspect of the present invention, said liposomal formulations comprises liposomes selected from the group of conventional liposomes (Tumori. 2003 May-June; 89(3):237-49; From conventional to stealth liposomes: a new frontier in cancer chemotherapy; Cattel Ll, Ceruti M, Dosio F.).

In the present invention, conventional liposomes are liposomes that consist of a phospholipid or of a phospholipid and a steroid, wherein said conventional liposomes may exist as monolayer or multilayer liposomes. Conventional liposomes may be prepared in a manner known by the person skilled in the art (e.g. Liposome: classification, preparation, and applications; Abolfazl, A. et al.; Nanoscale Research Letters 2013; 8:102).

A further aspect of the present invention is said liposomal formulation, wherein said phospholipid may be selected from the group comprising phosphotidylcholine, phosphotidylserine, phosphotidylethanolamine, phosphoinositol, 1,2-dilauroyl-sn-glycero-3-phosphocholine, 1,2-dioleoyl-sn-glycero-3[phospho-L-serine] sodium salt, dipalmitoylphosphotidylcholine, distearoylphosphotidylcholine, dipalmitoylphosphotidylserine, dipalmitoylphosphotidylglycerol, 1-stearoyl-2-linoleoyl-sn-glycero-3-[phosphor-L-serine] sodium salt, dioleoylphosphotidylcholine, and sphingomyelin. In formulations of the present invention, the phospholipid may be distearoylphosphotidylcholine.

A further aspect of the present invention is said liposomal formulation, wherein said steroid is added in order to prevent leaking of said pharmaceutically active ingredient and wherein said steroid may be selected from the group comprising cholesterol and its derivatives, including but not limited to cholesterol sulfate, cholesterol hemisuccinate and polymer-derivatized cholesterol and related sterols. Even another aspect of the present invention is said liposomal formulation, wherein said steroid is added in order to prevent leaking of said pharmaceutically active ingredient and wherein said steroid is cholesterol.

In a further aspect of the present invention, the ratio between above defined phospholipid and above defined steroid in said liposomal formulation may be in a range of 100/0 to 50/50 mol % of phospholipid and steroid respectively. In even another aspect of the present invention the ratio between above defined phospholipid and above defined steroid in said liposomal formulation may be 55/45 mol % of phospholipid and steroid respectively.

Liposomal formulations of the present invention may be prepared by conventional methods known to someone skilled in the art, these methods may be mechanical agitation, solvent evaporation, solvent injection and the surfactant solubilization method, wherein in one aspect of the invention said liposomes are prepared in a method of solvent evaporation, e.g. in a method of thin film hydration.

In said solvent evaporation method, the above defined lipid and steroid are dissolved in a common solvent in above defined ratio and the solvent is evaporated at reduced pressure (under vacuum). The remaining dry film is then hydrated with a buffer solution, promoting the formation of said liposomes. Said buffer solution may be selected from the group comprising ammonium sulfate buffer, sodium acetate buffer and calcium acetate buffer (pH gradient method).

The pharmaceutically active ingredients are loaded into said liposomes via a method of active or passive loading known to someone skilled in the art. In one aspect of the present invention, said method is a method of active loading, e.g. an active loading method induced by a trans-membrane gradient. Said trans-membrane gradient may be a trans-membrane ammonium gradient, induced by ammonium sulfate, a trans-membrane acetate gradient, induced by either calcium or sodium acetate or mixtures thereof, a trans-membrane pH gradient, induced by citrate or a gradient induced by manganese sulfate. In one embodiment of the present invention said gradient is a trans-membrane pH gradient induced by citrate.

In further embodiments of the invention, the encapsulation of the active compounds according to the present invention, e.g. compounds of formula I, e.g. compounds of formulae (I-a), (I-b), (I-c), (I-d), (I-e), (I-f), and (I-g), takes place at a pH allowing for maximum loading of the drug into liposomal vesicles. In some embodiments of the present invention, the preparation of compound-loaded liposomes occurs when the absolute charge of the active compounds is in a range between +0.5 and −0.5, e.g. it may be 0. Further, embodiments of the invention relate to processes for the preparation of said liposomes, wherein the pH value of the solution of the active compound for loading the vesicles is 2.0 to 9.0, for example the pH may be 4.6 to 8.2.

In processes for the preparation of the liposome formulations of the present invention, after hydration, the liposomes are extruded through a porous membrane in order to define their size range. The size of said liposomes may be in a range of 50-500 nm, e.g. in a size range of 130-150 nm.

The polydispersity index (PDI) of said liposomes prepared by above defined methods after above defined extrusion is below 0.100.

In order to set up the above defined trans-membrane gradient prior to encapsulation of the pharmaceutically active ingredient, the liposomes are dialyzed in a salt solution removing buffer in the liposomal dispersion, while keeping the buffer inside the liposomes.

For the purpose of encapsulation of pharmaceutically active ingredients into said liposomes, the pharmaceutically active ingredient is dissolved in the same salt solution as applied for above defined dialysis to establish the trans-membrane gradient. The solution of the pharmaceutically active ingredient is subsequently added to the liposomal dispersion. Loading of the drug occurs as a consequence of the trans-membrane gradient as is known to someone skilled in the art.

The liposomal formulations comprising at least one of compounds of formulae (I) and (I-a to I-g) that are for use according to the invention have a surprising pharmacological efficacy which could not have been predicted. The formulations of the present invention may comprise at least one part comprising one of the compounds of formulae (I-a) to (I-g), e.g. formula (I-g) and another part comprising another one of the compounds of formulae (I-a) to (I-g), i.e. the formulations may be mixed formulations comprising liposomes comprising at least two different beta-lactams as defined herein in individual liposomes, or at least two different beta-lactams as defined herein together in liposomal particles.

In another aspect of the present invention, said liposomal formulations with compounds according to formulae (I) and (I-a to I-g) as pharmaceutically active substances are therefore suitable for use as medicaments for the treatment and/or prophylaxis of diseases in humans and animals. In some embodiments of the medicaments for the treatment and/or prophylaxis of diseases in humans and animals, the at least one compound has formula (I-g). The liposomal formulations accoding to the present invention may be used in combination with at least one beta-lactamase-inhibitor (BLI), which may be administered separately. The BLI may also be formulated as liposomal drug, comprising said at least one BLI either alone or combined with the compounds of formulae (I-a) to (I-g), e.g. formula (I-g). Also contemplated are mixed liposomal formulations, wherein at least one part comprises at beta-lactam, e.g. compounds of formulae (I-a) to (I-g), e.g. formula (I-g) and another part of the formulations comprises another active compound, e.g. a BLI.

As used herein, a suitable BLI may be selected from the group comprising: clavulanic acid, tazobactam, sulbactam and other BLIs belonging to the groups of lactam inhibitors, DABCO inhibitors, BATSI inhibitors and/or metallo-beta-lactamase inhibitors. These BLIs together with the liposome formulations according to the present invention may be administered in methods of treatment or prevention and are compounds for the use in the treatment of prophylaxis of a subject having an infection caused by bacteria, especially gram-negative bacteria.

In yet another aspect of the invention, in the above mentioned formulations said formulation is further comprising a solubilizing agent, antioxidant, buffering agent, acidifying agent, complexation enhancing agent, saline, dextrose, lyophilizing aid, bulking agent, stabilizing agents, electrolyte, another therapeutic agent, alkalizing agent, antibacterial agents, antifungal agents, antiviral agents, antiparasitic agents, antimycotic agents, antimycobacterial agents, intestinal antiinfective agents, antimalaria agents, anti-inflammatory agents, anti-allergic agents, analgesic drugs, anaesthetic drugs, immunomodulators, immune suppressive agents, mono clonal antibodies, anti-neoplastic drugs, anti-cancer drugs, anti-emetics, antidepressivse, anti-psychotics, anxiolytics, anti-convulsives, HMG CoA reductase inhibitors and other anti-cholesterol agents, anti-hypertensives, Insulins, oral anti-diabetics, proton pump inhibitors, oral or parenteral anti coagulants, diuretics, digoxins, broncho dialators, antiarrythmics, vaso pressors, steroids and derivatives or combinations thereof.

In another aspect of the present invention, said liposomal formulations with compounds according to formulae (I) and (I-a to I-g) as pharmaceutically active substances are distinguished in particular by an advantageous range of antibacterial effects. In some embodiments of the compound in the liposomes has formula (I-g).

The present invention therefore further relates to the use of the liposomal formulations as defined above with compounds according to formulae (I) and (I-a to I-g) as pharmaceutically active substances for use according to the invention for the treatment and/or prophylaxis of diseases caused by bacteria, especially gram-negative bacteria.

The liposomal formulations as defined above with compounds according to formulae (I) and (I-a to I-g) as pharmaceutically active substances of the invention exhibit an antibacterial spectrum against gram-negative bacteria and selected gram-positive bacteria combined with low toxicity.

Liposomal formulations with compounds according to formulae (I) and (I-a to I-g) as pharmaceutically active substances according to the invention are particularly useful in human and veterinary medicine for the prophylaxis and treatment of local and systemic infections which are caused for example by the following pathogens or by mixtures of the following pathogens:

Aerobic gram-positive bacteria including but not limited to Staphylococcus spp. (S. aureus), Streptococcus spp. (S. pneumoniae, S. pyogenes, S. agalactiae, Streptococcus group C and G) as well as Bacillus spp. and Listeria monocytogenes;

Aerobic gram-negative bacteria: Enterobacteriaceae including but not limited to Escherichia spp. (E. coli), Citrobacter spp. (C. freundii, C. diversus), Klebsiella spp. (K. pneumoniae, K. oxytoca), Enterobacter spp. (E. cloacae, E. aerogenes), Morganella morganii, Hafnia alvei, Serratia spp. (S. marcescens), Proteus spp. (P. mirabilis, P. vulgaris, P. penneri), Providencia spp. (P. stuartii, P. rettgeri), Yersinia spp. (Y. enterocolitica, Y. pseudotuberculosis), Salmonella spp., Shigella spp. and also non-fermenters including but not limited to Pseudomonas spp. (P. aeruginosa), Burkholderia spp. (B. cepacia), Stenotrophomonas maltophilia, and Acinetobacter spp. (A. baumannii, Acinetobacter gen. sp. 13TU, Acinetobacter gen. sp. 3) as well as Bordetella spp. (B. bronchiseptica), Moraxella catarrhalis and Legionella pneumophila; furthermore, Aeromonas spp., Haemophilus spp. (H. influenzae), Neisseria spp. (N. gonorrhoeae, N. meningitidis) as well as Alcaligenes spp. (including A. xylosoxidans), Pasteurella spp. (P. multocida), Vibro spp. (V. cholerae), Campylobacter jejuni and Helicobacter pylori.

Moreover, the antibacterial spectrum also covers strictly anaerobic bacteria including but not limited to Bacteroides spp. (B. fragilis), Peptostreptococcus spp. (P. anaerobius), Prevotella spp., Brucella spp. (B. abortus), Porphyromonas spp., and Clostridium spp. (Clostridium perfringens).

The above listing of pathogens is merely exemplary and in no way to be regarded as limiting. Examples of diseases which may be caused by the said pathogens and which may be prevented, improved or cured by the liposomal formulations with compounds according to formulae (I) and (I-a to I-g) as pharmaceutically active substances according to the invention are, for example:

Respiratory tract infections such as lower respiratory tract infections, lung infection in cystic fibrosis patients, acute exacerbation of chronic bronchitis, community acquired pneumonia (CAP), nosocomial pneumonia (including ventilator-associated pneumonia (VAP)), diseases of the upper airways, diffuse panbronchiolitis, tonsillitis, pharyngitis, acute sinusitis and otitis including mastoiditis; urinary tract and genital infections for example cystitis, uretritis, pyelonephritis, endometritis, prostatitis, salpingitis and epididymitis; ocular infections such as conjunctivitis, corneal ulcer, iridocyclitis and post-operative infection in radial keratotomy surgery patients; blood infections, for example septicaemia; infections of the skin and soft tissues, for example infective dermatitis, infected wounds, infected burns, phlegmon, folliculitis and impetigo; bone and joint infections such as osteomyelitis and septic arthritis; gastrointestinal infections, for example dysentery, enteritis, colitis, necrotising enterocolitis and anorectal infections; intraabdominal infections such as typhoid fever, infectious diarrhea, peritonitis with appendicitis, pelviperitonitis, and intra-abdominal abscesses; infections in the oral region for example infections after dental operations; other infections for example, meliodosis, infectious endocarditis, hepatic abscesses, cholecystitis, cholangitis, mastitis as well as meningitis and infections of the nervous systems.

In addition to humans, bacterial infections can also be treated in animals, such as primates, pigs, ruminants (cow, sheep, goat), horses, cats, dogs, poultry (such as hen, turkey, quail, pigeon, ornamental birds) as well as productive and ornamental fish, reptiles and amphibians.

The liposomal formulations with compounds according to formulae (I) and (I-a to I-g) as pharmaceutically active substances according to the invention may act systemically and/or locally. They can for this purpose be administered in a suitable way, such as, for example, perorally, parenterally, sub-cutaneously, intravenously, pulmonarily, intrathecally nasally, sublingually, lingually, buccally, rectally, dermally, transdermally, conjunctivally, otically or as an implant or stent.

For these administration routes the liposomal formulations of the invention can be administered in suitable administration forms.

### EXAMPLES

1. Preparation of Liposomes

All liposomal formulations were prepared by thin film hydration method. Briefly, accurately weighed amounts of DSPC/Chol: 55/45 (mol) were dissolved in a round bottomed flask with chloroform and a film was created under reduced pressure at 40° C. After complete removal of the organic solvent the film was hydrated with one of the following buffers: 300 mM Ammonium sulphate; 150 mM Sodium acetate; 120 mM Calcium acetate, or 0.9% NaCl (used as negative control), and HEPES/saline buffer (HBS) pH 7.4 (used as negative control) at a final concentration of 60 mM Lipid.

After one hour of hydration (complete removal of the film from the flask's walls) at 65° C., the liposomes were extruded using a Lipex thermobarrel extruder equipped with polycarbonate membranes of various pore sizes (400, 200 and 100 nm) in order to reach a final size of 130-150 nm and PdI<0.100. At this size range the maximum theoretical encapsulation can be achieved. After extrusion, the liposomes were dialyzed against 0.9% NaCl (154 mM) in order to create a transmembrane gradient. To ensure complete exchange of the external buffer, 10 volume exchanges were done. Liposomes were stored at 2-8° C. and were diluted prior to use at the desired concentration each time using 0.9% NaCl.

Preparation of Beta-Lactam (Compound I-g) Solution

Accurately weighed amounts of Compound I-g were placed gradually to 0.9% NaCl and mixed until dissolved. Dissolution was aided by heating at 55° C.

Size Measurements of Liposomes

Measurements for the determination of liposome size were performed by Dynamic-LaserLight-Scattering (DLS) using a Malvern Nano ZS (224/SOP/002). This system is equipped with a 4 mW Helium/Neon Laser at 633 nm wavelength and measures the liposome samples with the non-invasive backscatter technology at a detection angle of 173°. Liposomes were diluted in aqueous phase to reach optimal liposome concentration and the experiments were carried out at 25° C.

Quantification of Compound I-g

Quantification of Compound I-g was done with Spectrophotometer at λₘₐₓ=280 nm. A calibration curve was prepared (R²=0.9998) and according to this, all spectrophotometer readings were calculated to concentration. Additionally, the same procedure was followed for quantification of the substances used as positive control. All samples were prepared using the same matrix (25% phys. NaCl solution (0.9%)/75% 2-propanol: vol/vol).

Evaluation of the Analytic Method Used for the Quantification of Compound I-g

In order to ensure correct quantification of Compound I-g, several trials were done. Initially, plain liposomes at various lipid concentrations were diluted and measured. The absorbance given at this wavelength is considered as negligible. Nevertheless it is confirmed that liposomes do not interfere with the OD signal. Furthermore, the absorbance of a freshly prepared solution of 2.5 mM (in NaCl 0.9%) of Compound I-g was measured either as a plain solution or in combination with liposomes. The solution was diluted in three different ways.

1. In the first case, the solution was diluted first with 0.9% NaCl (2, 4, 6 times) and then aliquot of it (200 μl) was withdrawn and mixed with 2-propanol (600 μl) in order to result in a mixture of 25% phys. NaCl-solution (0.9%)/75% 2-propanol.

2. In the second case, the solution (2.5 mM) was first mixed with 10 mM liposomal dispersion (inside calcium acetate/outside 0.9% NaCl) in ratio 1:1 (vol/vol), an aliquot (25 μl) of this mixture was withdrawn and diluted with 0.9% NaCl (175 μl) (stock solution) and to this solution was added 2-propanol (600 μl) in order to result in the same final matrix (25% phys. NaCl-solution (0.9%)/75% 2-propanol). Additionally, in order to scan the whole spectrum between 0.1 and 0.7, several dilution steps were performed at the stock solution and measured using the same sample preparation steps.

3. In the third case, solution of compound (I-g) (2.5 mM) was mixed with 10 mM liposomal dispersion (inside calcium acetate/outside 0.9% NaCl) in ratio 1:1 (vol/vol) and was further diluted 24, 48 and 96 times using at first 0.9% NaCl solution (500, 1100, and 2300 μl) and then 2-propanol (1800, 3600 and 7200 μl). The aim was to investigate the range of the OD within which the calibration curve is reliable. All sample preparation methods resulted in a deviation from the theoretical value. However, the third sample preparation procedure showed the least deviation from the theoretical value. A deviation of 15-20% in a feasibility study is acceptable, taking into consideration that an efficient active loading should results in more than 80-90% of drug encapsulation.

Detection of the Encapsulated Drug

In order to calculate the loading efficiency, drug-loaded liposomes were separated from the free drug using centrifugal ultrafiltration. Centrifugal ultrafiltration was performed using centrisart tubes (ultrafiltration concentrators) equipped with 100 kD PES membranes. Briefly, specific amount of sample was pipetted into the centrisart tube and it was placed for centrifugation in order to separate liposomes from their external aqueous medium. With this method, liposomes form sediment on the bottom of the tube while in the ultrafiltrate is collected the external medium containing the free Compound I-g. Following, the concentration of Compound I-g in the ultrafiltrate was measured in the spectrophotometer as described above and the percentage of encapsulation was calculated according to the following formula:

E %=[Compound I-g total]−[Compound I-g−filtrate]/[Compound I-g−total]%

1. A liposomal pharmaceutical formulation comprising a compound according to formula (I) as active ingredient, 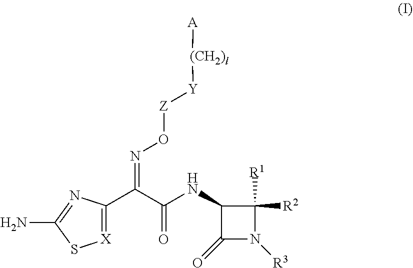
characterized in that
R¹ and R² represent methyl,
R³ represents —O—(SO₂)OH,
X represents CH,
Z represents a two carbon alkyl-chain, substituted with a carboxy substituent,
Y represents O,
represents 0
A represents phenyl substituted with a substituent of the following formula 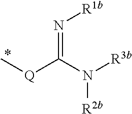
wherein
R^{1b} and R^{2b} represent hydrogen,
R^{3b} represents aminoethyl, azetidine, pyrrolidine or piperidine,
Q represents a bond,
* is the linkage site to the residue represented by A, and
and the salts thereof, the solvates thereof and the solvates of the salts thereof,
wherein the liposomes comprise at least one phospholipid and one steroid.

2. The liposomal pharmaceutical formulation according to claim 1, wherein the active ingredient is at least one member of the group of compounds selected from formulae (I-a) to (I-g):

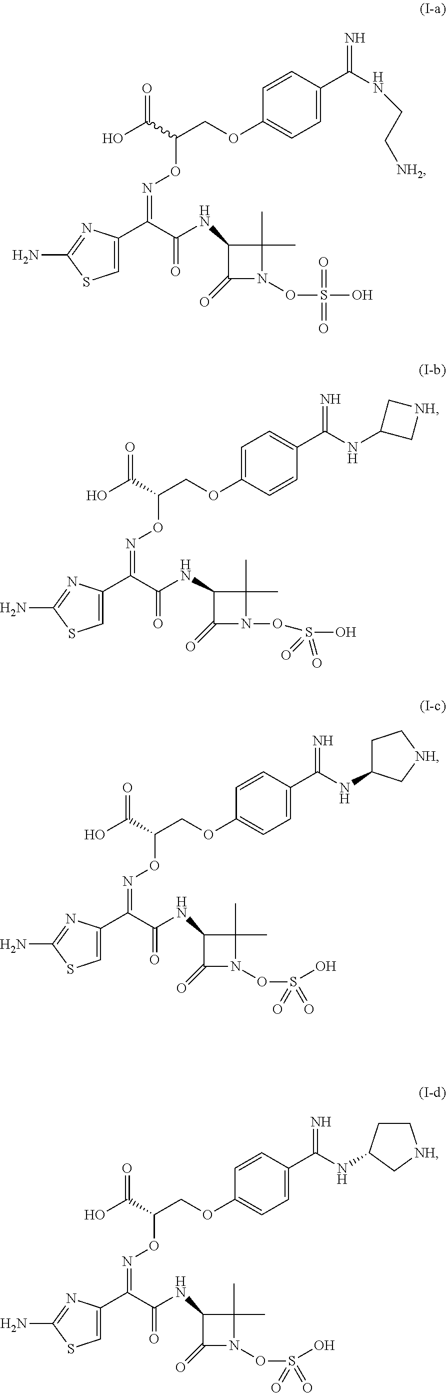 [SMILES: CC1(C)[C@H](NC(=O)/C(=N\OC(COc2ccc(C(=N)NCCN)cc2)C(=O)O)c2csc(N)n2)C(=O)N1OS(=O)(=O)O ; CC1(C)[C@H](NC(=O)/C(=N\O[C@@H](COc2ccc(C(=N)NC3CNC3)cc2)C(=O)O)c2csc(N)n2)C(=O)N1OS(=O)(=O)O ; CC1(C)[C@H](NC(=O)/C(=N\O[C@@H](COc2ccc(C(=N)N[C@H]3CCNC3)cc2)C(=O)O)c2csc(N)n2)C(=O)N1OS(=O)(=O)O ; CC1(C)[C@H](NC(=O)/C(=N\O[C@@H](COc2ccc(C(=N)N[C@@H]3CCNC3)cc2)C(=O)O)c2csc(N)n2)C(=O)N1OS(=O)(=O)O]

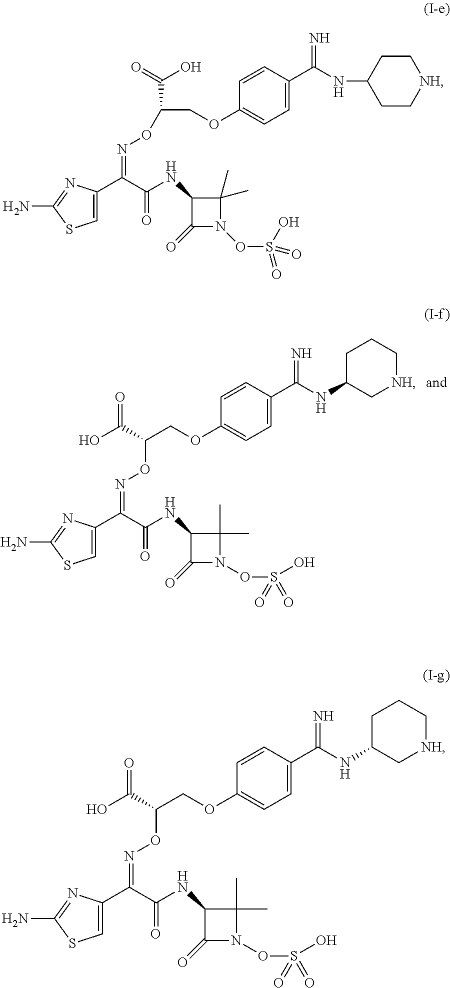 [SMILES: CC1(C)[C@H](NC(=O)/C(=N\O[C@@H](COc2ccc(C(=N)NC3CCNCC3)cc2)C(=O)O)c2csc(N)n2)C(=O)N1OS(=O)(=O)O ; CC1(C)[C@H](NC(=O)/C(=N\O[C@@H](COc2ccc(C(=N)N[C@H]3CCCNC3)cc2)C(=O)O)c2csc(N)n2)C(=O)N1OS(=O)(=O)O ; CC1(C)[C@H](NC(=O)/C(=N\O[C@@H](COc2ccc(C(=N)N[C@@H]3CCCNC3)cc2)C(=O)O)c2csc(N)n2)C(=O)N1OS(=O)(=O)O]

3. The liposomal pharmaceutical formulation according to claim 1, wherein the active ingredient is a compound of formula (I-g).

4. The liposomal pharmaceutical formulation according to claim 1, wherein the phospholipid is selected from the group comprising phosphotidylcholine, phosphotidylserine, Phosphotidylethanolamine, phosphoinositol, 1,2-dilauroyl-sn-Glycero-3-Phosphocholine, 1,2-dioleoyl-sn-Glycero-3 [Phospho-L-Serine] sodium salt, dipalmitoylphosphotidylcholine, distearoylphosphotidylcholine, dipalmitoylphosphotidylserine, dipalmitoylphosphotidylglycerol, 1-stearoyl-2-linoleoyl-sn-glycero-3-[phosphor-L-serine] sodium salt, dioleoylphosphotidylcholine, and sphingomyelin.

5. The liposomal pharmaceutical formulation according to claim 1, wherein the steroid is selected from the group comprising cholesterol, derivatives of cholesterol, and polymer-derivatized cholesterol.

6. The liposomal pharmaceutical formulation according to claim 1, wherein the phospholipid is distearoylphosphotidylcholine.

7. The liposomal pharmaceutical formulation according to claim 1, wherein the steroid is cholesterol.

8. The liposomal pharmaceutical formulation according to claim 1, wherein the ratio of phospholipid to steroid is in the range from 60:40 to 40:60.

9. The liposomal pharmaceutical formulation according to claim 1, wherein the ratio of phospholipid to steroid is 55:45.

10. The liposomal pharmaceutical formulation according to claim 1, wherein the lipid to drug ratio is in the range of 1:0.1 to 1:0.5.

11. The liposomal pharmaceutical formulation according to claim 1, wherein the lipid to drug ratio is 1:0.3.

12. The liposomal pharmaceutical formulation according to claim 1, for use in the treatment and/or prevention of bacterial infections.

13. A method of treatment and/or prevention of bacterial infection comprising administering a lipid formulation according to claim 1.
